(19) United States
(12) Patent Application Publication
(10) Pub. No.: US 2010/0100142 A1
(43) Pub. Date: Apr. 22, 2010

(54) INTRODUCER WITH DISSECTION FUNCTION FOR PECTUS EXCAVATUM REPAIR OPERATION

(76) Inventor: Hyung Joo Park, Seoul (KR)

Correspondence Address: CHRISTOPHER PAUL MITCHELL, 888 16TH ST., NW, SUITE 800, WASHINGTON, DC, 20006, US

(21) Appl. No.: 12/531,693

(22) PCT Filed: Aug. 24, 2007
(86) PCT No.: PCT/KR07/04053

§ 371 Date: Sep. 16, 2009

(30) Foreign Application Priority Data
Mar. 19, 2007 (KR) 10-2007-0026464

(51) Int. Cl. A61B 17/56

(52) U.S. Cl. 606/86 R

(57) ABSTRACT

Disclosed herein is an introducer with a dissection function for correction of pectus excavatum, which can form a passageway in the chest to insert a correcting metal bar thereinto in an operation to correct pectus excavatum, which is a sort of chest deformity. The introducer includes: an induction pipe having a hollow cross-section and made of a rigid material, the induction pipe being curved longitudinally; and an observation part joined to an end portion of the induction pipe, the observation part having a hollow cross-section and a transparent wall body.

### TECHNICAL FIELD

The present invention relates to an introducer with a dissection function for correction of pectus excavatum, and more particularly, to an introducer for forming a passageway in the chest to insert a correcting metal bar thereinto in an operation to correct pectus excavatum, which is a sort of chest deformity.

### BACKGROUND ART

The deformities of the chest are largely divided into pectus excavatum called funnel chest and pectus carinatum called pigeon chest. Causes of the deformities are not yet exactly clarified, but it has been known there are some genetic effects. As shown in FIG. 1, the breastbone is formed in such a way that the ribs (a), the sternum (b), the costal cartilages (c) and the vertebrae are connected with one another, and pectus excavatum and pectus carinatum are mostly generated not by deformity of the ribs (a) but by abnormality in the costal cartilages (C), which construct the front of the chest and connect the sternum and the ribs with each other. The degree of concaveness or convexness is not severe at the time of a birth, but may get more severe while a patient grows up.

Particularly, in case of pectus excavatum, hollowed chest walls press the heart or the lungs to thereby cause a functional disorder and deteriorate motor functions. Since children may frequently take a cold and have symptoms of pneumonia, it may make the respiratory organs worse or retard the growth thereof. Moreover, children of tender age or teenagers may meet mental and emotional troubles.

There is a Ravitch operation as a surgical operation method to correct pectus excavatum. The Ravitch operation is performed by the steps of largely opening the anterior chest and removing all of cartilages. Accordingly, the Ravitch operation has a problem in that soft motor functions of the chest since the chest walls get weak or are adhered after the operation, and in that a big scar remains on the chest.

In 1997, Donald Nuss from United States invented a new operation method (called ‘Nuss operation’) to correct pectus excavatum instead of the Ravitch operation.

The Nuss operation is performed by the steps of incising areas below both armpits of a patient, whose chest walls are hollowed as indicated by a hatched line of FIG. 2(a), to about 1˜2 cm, inserting a curved correcting metal bar 400 into the cut portions, and rotating the correcting metal bar 400 as shown in FIG. 2(b) to lift up a hollowed bone, whereby it can make a normal chest form.

The Nuss operation has several advantages in that it leaves a scar of only about 1˜2 cm on both sides of the chest in comparison with the Ravitch operation, in that the patient can keep flexibility and resilience since it can correct the deformed chest into the normal chest without resection of the costal cartilages, in that it takes short time to perform the operation, and in that it causes less bleeding.

However, in the Nuss operation, a doctor pushes the correcting metal bar from a side of one chest to the inside of the thoracic cavity, and then, draws out the correcting metal bar toward a side of the other chest. In this instance, the Nuss operation also has a problem in that the doctor has to perform the operation according to his or her experiences since the doctor cannot observe the inside of the thoracic cavity while the correcting metal bar passes through the inside of the thoracic cavity. That is, in the learned world, there are several reports of examples that patients encountered danger since the correcting metal bar excessively pressed the internal organs directly connected to the patient's life, such as the heart, the lungs, the great arteries, and so on, inside the thoracic cavity to thereby cause damages or bleeding thereof. Moreover, the Nuss operation has another problem in that it is very difficult to draw out the correcting metal bar from the side of the opposite chest.

### DISCLOSURE OF INVENTION

### Technical Problem

Accordingly, the present invention has been made in an effort to solve the abovementioned problems occurring in the prior arts, and it is an object of the present invention to provide an introducer with a dissection function for correction of pectus excavatum, which can previously secure a path through which a correcting metal bar can pass the inside of the thoracic cavity while a doctor observes the inside of the thoracic cavity with an endoscope in the Nuss operation, thereby preventing bleeding of the inside of the thoracic cavity or damages of the internal organs, and allowing the doctor to promptly check whether or not the internal organs are damaged.

Another object of the present invention is to provide an introducer with a dissection function for correction of pectus excavatum, which allows the doctor to easily draw out the introducer from the thoracic cavity since the doctor can check positions of operating instruments, such as forceps, drawn out from the patient's opposite chest through the endoscope.

### Technical Solution

To achieve the above objects, the present invention provides an introducer with a dissection function for correction of pectus excavatum, the introducer comprising: an induction pipe having a hollow cross-section and made of a rigid material, the induction pipe being curved longitudinally; and an observation part joined to an end portion of the induction pipe, the observation part having a hollow cross-section and a transparent wall body.

It is preferable that the induction pipe is made of a transparent material.

It is also preferable that the observation part is made of a flexible material with elasticity.

Furthermore, it is preferable that the other end portion of an opposite side to one end portion of the observation part, which is joined to the induction pipe, is opened.

Moreover, it is also preferable that the other end portion of an opposite side to one end portion of the observation part, which is joined to the induction pipe, has a pointed tip.

### Advantageous Effects

According to the present invention, the introducer for correction of pectus excavatum can previously secure the path, through which the correcting metal bar can pass, in the Nuss operation, prevent bleeding inside the thoracic cavity or damages of the internal organs during the process to secure the path, and allow the doctor to promptly check whether or not the internal organs are damaged. Furthermore, the introducer for correction of pectus excavatum according to the present invention can reduce an operation period, be used disposably since its manufacturing cost is inexpensive, and allow the doctor to easily draw out the introducer from the thoracic cavity since the doctor can exactly grasp a portion of the opposite chest from which the introducer will be drawn out.

### BRIEF DESCRIPTION OF THE DRAWINGS

FIG. 1 is a view showing a structure of bones constituting the chest.

FIG. 2 is a schematic view of the principle of Nuss operation.

FIG. 3 is a view showing an introducer according to the present invention.

FIG. 4 is a perspective view of an endoscope.

FIG. 5 is a perspective view of a correcting metal bar.

FIG. 6 is a view showing a form of an observation part according to the present invention.

FIG. 7 is a view showing a state where a surgical operation is performed using the introducer of the present invention.

FIG. 8 is a view showing a state where the introducer and the correcting metal bar are connected with each other.

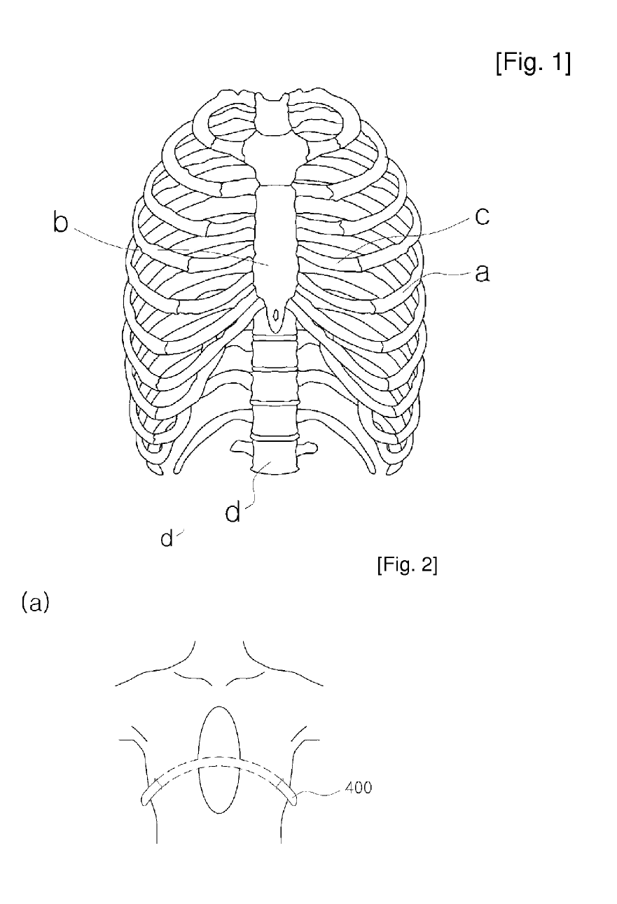

### EXPLANATION ON REFERENCE NUMERALS OF MAIN ELEMENTS IN DRAWINGS

1: introducer 100: induction pipe 200: observation part 300: endoscope 400: correcting metal bar 500: connection tube

### MODE FOR THE INVENTION

Reference will be now made in detail to the preferred embodiment of the present invention with reference to the attached drawings.

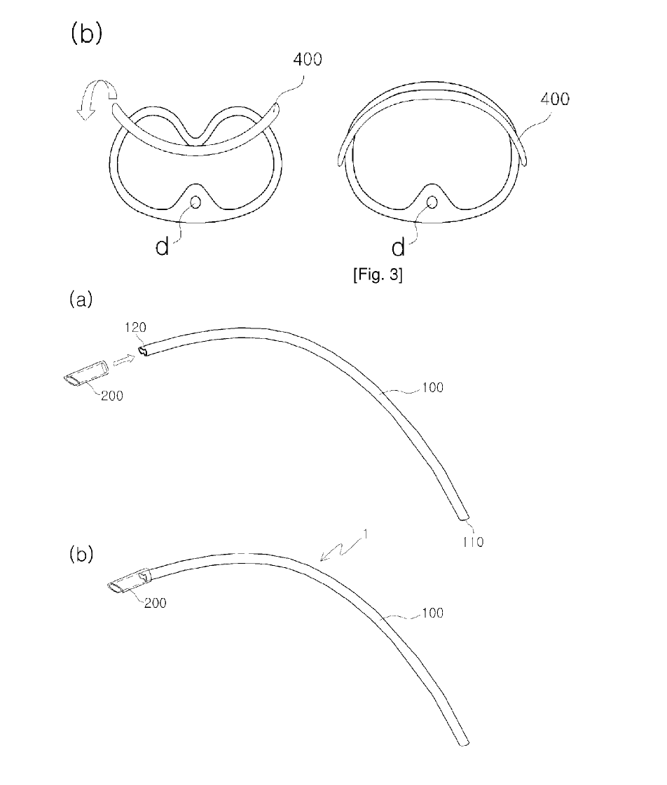

FIG. 3 illustrates a form of an introducer according to the present invention.

The introducer 1 includes: an induction pipe 100 having a hollow cross-section and made of a rigid material, the induction pipe 100 being curved longitudinally; and an observation part 200 joined to an end portion of the induction pipe 100 and having a hollow cross-section, the observation part 200 having a transparent wall body.

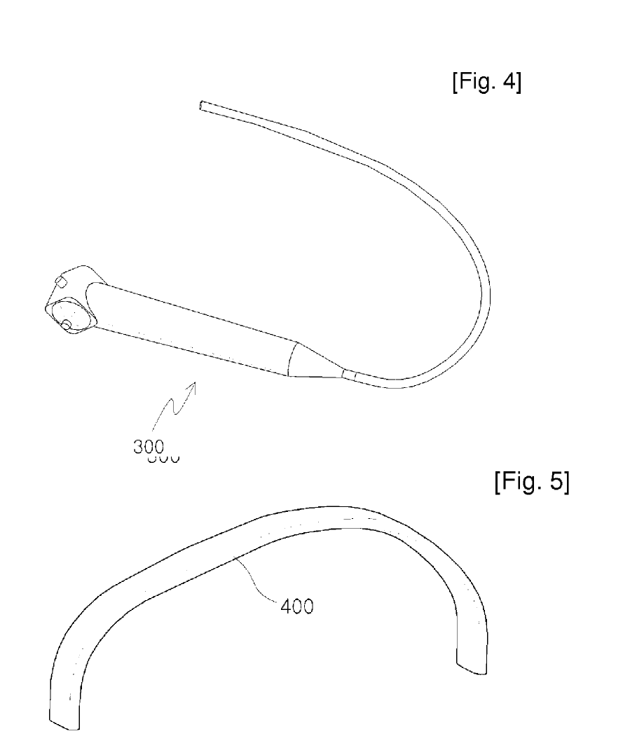

The induction pipe 100 has an inlet 110 for inserting the endoscope 300, shown in FIG. 4, thereinto and an outlet 120, to which the observation part 200 is attached. The induction pipe 100 has the hollow section since it has a through hole perforating it.

If the endoscope 300 inserted into the introducer can freely go into the introducer since an inserted portion thereof is flexible, any kind of the endoscope can be used.

It is preferable that the induction pipe 100 is made of rigid metal to transfer power to go into the thoracic cavity. Moreover, the induction pipe 100 is longitudinally curved, and in this instance, it is preferable that the induction pipe 100 is curved in a form substantially similar to a correcting metal bar 400 shown in FIG. 5. Additionally, an induction pipe constructed in such a way that it can be freely bent by a doctor when it is used after being manufactured in a straight form also belongs to the scope of the present invention.

The observation part 200 having the transparent wall body is attached to the outlet 120 of the induction pipe 100, so that the endoscope inserted into the induction pipe informs the doctor of information, such as positions of the internal organs, inside the thoracic cavity through the observation part having the transparent wall body.

As shown in FIG. 3, it is preferable that an end portion of the outlet 120 of the induction pipe 100 to which the observation part 200 is attached is tiered or inclined so that the observation part can be easily attached thereto.

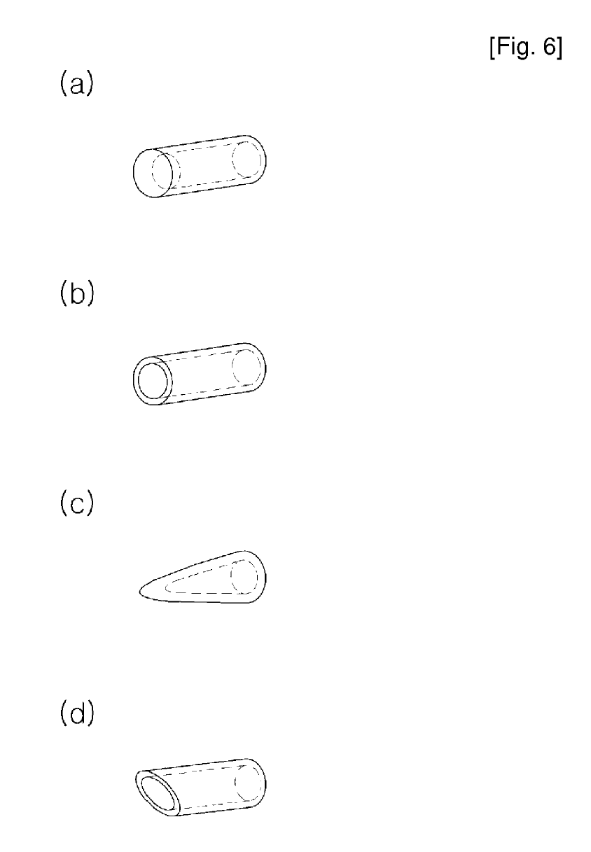

Furthermore, the other end portion of the observation part, to which the induction pipe is not attached, is stopped by a transparent wall body as shown in FIG. 6(a) or opened as shown in FIG. 6(b). Moreover, as shown in FIGS. 6(c) and (d), the opposite end portion of the observation part may be formed in a cone shape or inclined. As described above, when the introducer goes into the thoracic cavity, the observation part allows the doctor to observe the inside of the thoracic cavity and guides the introducer while dissecting tissues between various internal organs inside the thoracic cavity.

In order to correct pectus excavatum, first, incise the skin of both sides of the chest of a patient, who has pectus excavatum, to a length of about 1˜2 cm, and bore a hole in the chest wall to insert the introducer into the thoracic cavity.

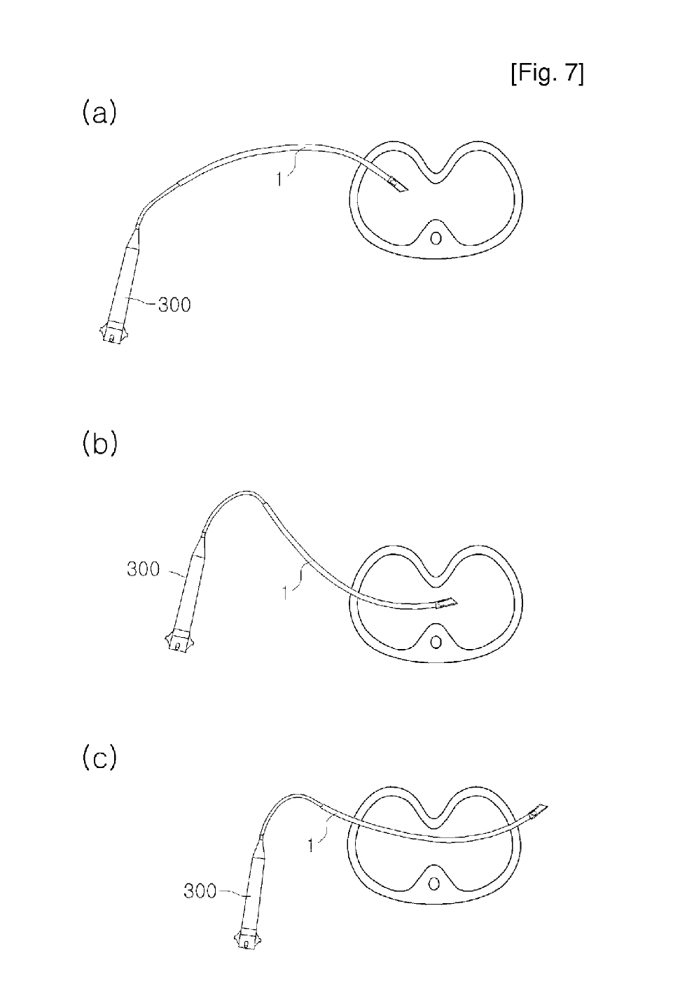

Next, as shown in FIG. 7(a), insert a front end of the observation part of the introducer into the thoracic cavity, insert the endoscope into the induction pipe through the inlet of the induction pipe till it reaches the observation part to check positions of the internal organs inside the thoracic cavity.

Thereafter, as shown in FIG. 7(b), in the state where the endoscope is located inside the observation part, the doctor can regulate an entering direction of the introducer and dissect the tissues between the internal organs while observing the positions of the internal organs inside the thoracic cavity after inserting the introducer including the endoscope into the thoracic cavity.

In addition, the introducer can safely go into an incised portion of the other side of the chest after crossing the thoracic cavity while dissecting the tissues between the internal organs and the chest wall using the front end of the observation part shown in FIGS. 6(c) and (d). It is preferable that the observation part is made of flexible synthetic material having elasticity. The reason is that the observation part must be restored to its original state by elasticity and not damage the internal organs.

When the introducer reaches the incised portion of the opposite side of the chest, the doctor bores a hole in the chest wall of the incised portion, inserts the forceps into the hole, and picks up the observation part with the forceps to draw out the introducer as shown in FIG. 7(c).

In this instance, since the endoscope is inserted into the introducer to allow the doctor to secure a visual field through the observation part, the doctor can check that the forceps inserted into the hole bored in the chest wall grasp the front end of the observation part of the introducer through the endoscope, whereby the doctor can perform the operation easily.

As described above, the induction pipe of the introducer may be made of a rigid material such as metal, but it is also preferable that it is made of synthetic resin, which can provide rigidity and transparency of the wall body.

In this case, the doctor can observe bleeding inside the thoracic cavity or the state of the internal organs while entering and backing only the endoscope along the induction pipe penetrating between the chest walls.

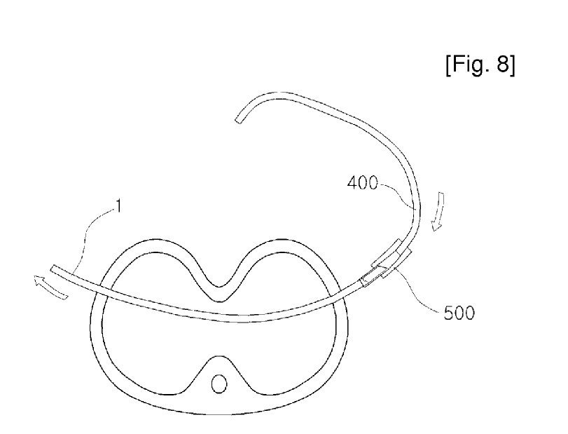

When the introducer protrudes to the outside of the patient's body after penetrating the thoracic cavity, as shown in FIG. 8, in a state where the observation part is removed or left as it is, a connection tube 500 is fit to the front end of the introducer 1 and the correcting metal bar 400 is fit to the connection tube 500, so that the correcting metal bar 400 is connected to the introducer 1.

Next, when the doctor pulls the introducer at the side of the chest to which the introducer 1 is inserted and pushes the correcting metal bar 400 at the opposite side, the introducer is drawn out through the incised portion to which the introducer is first inserted, and finally, the correcting metal bar is also mounted through the inside of the thoracic cavity in such a way that both end portions of the correcting metal bar protrude outwardly from the thoracic cavity. In this instance, like the general Nuss operation, the correcting metal bar is rotated to lift up the hollowed portion of the chest, whereby correction of pectus excavatum can be achieved.

Since a hollowed form of pectus excavatum may be asymmetric, in case where the correcting metal bar is mounted in the patient's chest after being manufactured according to the hollowed form of the chest, there is a problem in that it is difficult to guess where a front end of the correcting metal bar is located inside the thoracic cavity since the form of the correcting metal bar is varied. However, if the induction pipes of the introducers according to the present invention are formed uniformly and mass-produced, the doctor can guess where the front end of the introducer is located in consideration of inserted level and direction of the induction pipe, whereby the doctor can perform the operation safely.

The introducer according to the present invention can be easy to manufacture, reduce manufacturing expenses, and be used disposably since its structure is simple.

Post-processing of fixing the correcting metal bar mounted inside the thoracic cavity to the ribs and suturing the incised portion will not be described since it is not related with the technical idea of the present invention.

### INDUSTRIAL APPLICABILITY

As described above, according to the present invention, the introducer for correction of pectus excavatum can previously secure the path, through which the correcting metal bar can pass, in the Nuss operation, prevent bleeding inside the thoracic cavity or damages of the internal organs during the process to secure the path, and allow the doctor to promptly check whether or not the internal organs are damaged, whereby the doctor can perform the operation in safe.

1. An introducer with a dissection function for correction of pectus excavatum comprising:
an induction pipe having a hollow cross-section and made of a rigid material, the induction pipe being curved longitudinally; and
an observation part joined to an end portion of the induction pipe, the observation part having a hollow cross-section and a transparent wall body.

2. The introducer according to claim 1, wherein the induction pipe is made of a transparent material.

3. The introducer according to claim 1, wherein the observation part is made of a flexible material with elasticity.

4. The introducer according to claim 1, wherein the other end portion of an opposite side to one end portion of the observation part, which is joined to the induction pipe, is opened.

5. The introducer according to claim 1, wherein the other end portion of an opposite side to one end portion of the observation part, which is joined to the induction pipe, has a pointed tip.
